(19) United States
(12) Patent Application Publication
(10) Pub. No.: US 2019/0374129 A1
(43) Pub. Date: Dec. 12, 2019

(54) ENDOSCOPE WITH INTEGRAL NAVIGATION SENSOR

(71) Applicants: Acclarent, Inc., Irvine, CA (US); Biosense Webster (Israel) Ltd., Yokneam (IL)

(72) Inventors: Jetmir Palushi, Irvine, CA (US); Fatemeh Akbarian, Rancho Palos Verdes, CA (US); Behnam Amin, Mission Viejo, CA (US); Ehsan Shameli, Irvine, CA (US); Athanasios Papadakis, Newport Beach, CA (US); Assaf Govari, Haifa (IL); Yehuda Algawi, Binyamina (IL); Vadim Gliner, Haifa (IL)

(21) Appl. No.: 16/002,016

(22) Filed: Jun. 7, 2018

(51) Int. Cl. A61B 5/06; A61B 1/00; A61B 1/06; A61B 1/005; A61B 1/233; A61B 34/20

(52) U.S. Cl. CPC A61B 5/062; A61B 1/00101; A61B 1/0661; A61M 25/0127; A61B 1/233; A61B 34/20; A61B 2034/2051; A61B 1/0052

(57) ABSTRACT

An apparatus includes an endoscope, a navigation sensor, and an interface feature. The endoscope includes a body, a shaft extending distally from the body, and a window located at a distal portion of the shaft. The navigation sensor is position at the distal portion of the shaft. The interface feature can couple the navigation sensor with an image guidance system. The navigation sensor can cooperate with the image guidance system to provide feedback indicating a position of the navigation sensor in three-dimensional space.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MM, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of medical procedures where anatomical landmarks are not present or are difficult to visualize endoscopically.

While several systems and methods have been made and used in medical procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary sinus surgery navigation system being used on a patient seated in an exemplary medical procedure chair;

FIG. 2 depicts a perspective view of an exemplary endoscope suitable for use with the sinus surgery navigation system of FIG. 1;

FIG. 3 depicts a cross-sectional view of a distal end of a flexible shaft of the endoscope of FIG. 2, taken along line 3-3 of FIG. 2;

FIG. 4 depicts a perspective view of another exemplary endoscope suitable for use with the sinus surgery navigation system of FIG. 1; and

FIG. 5 depicts a cross-sectional view of a distal end of a flexible shaft of the endoscope of FIG. 4, taken along line 5-5 of FIG. 4.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Exemplary Image Guided Surgery Navigation System

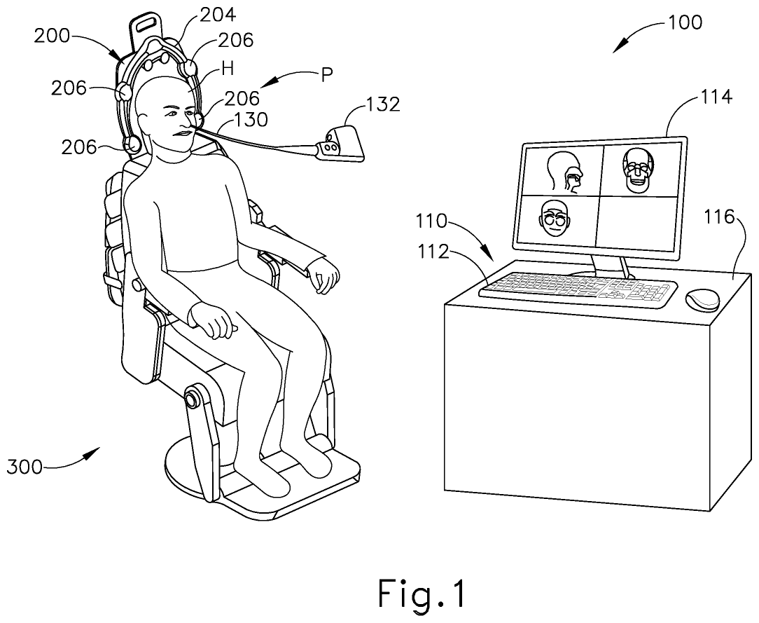

FIG. 1 shows an exemplary IGS navigation system (100) enabling a medical procedure to be performed using image guidance. In some instances, IGS navigation system (100) is used during a procedure where a dilation instrument assembly (not shown) is used to dilate the ostium of a paranasal sinus; or to dilate some other anatomical passageway (e.g., within the ear, nose, or throat, etc.). As another merely illustrative example, IGS navigation system (100) may be used during performance of any other kind of medical procedure within a patient's head, including but not limited to within the patient's nasal cavity, paranasal sinuses, Eustachian tubes, etc.; elsewhere within a patient's head; within a patient's throat; or elsewhere within a patient's body. Various suitable locations and clinical contexts in which IGS navigation system (100) may be used will be apparent to those of ordinary skill in the art in view of the teachings herein.

In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pub. No. 2016/0310042, entitled “System and Method to Map Structures of Nasal Cavity,” published Oct. 27, 2016; and U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein.

IGS navigation system (100) of the present example comprises a field generator assembly (200), which comprises set of magnetic field generators (206) that are integrated into a horseshoe-shaped frame (204). Field generators (206) are operable to generate alternating magnetic fields of different frequencies around the head of the patient. Field generators (206) thereby enable tracking of the position of a navigation guidewire (130) that is inserted into the head of the patient. Various suitable components that may be used to form and drive field generators (206) will be apparent to those of ordinary skill in the art in view of the teachings herein.

In the present example, frame (204) is mounted to a chair (300), with the patient (P) being seated in the chair (300) such that frame (204) is located adjacent to the head (H) of the patient (P). By way of example only, chair (300) and/or field generator assembly (200) may be configured and operable in accordance with at least some of the teachings of U.S. patent application Ser. No. 15/933,737, entitled “Apparatus to Secure Field Generating Device to Chair,” filed Mar. 23, 2018, the disclosure of which is incorporated by reference herein.

IGS navigation system (100) of the present example further comprises a processor (110), which controls field generators (206) and other elements of IGS navigation system (100). For instance, processor (110) is operable to drive field generators (206) to generate electromagnetic fields; and process signals from navigation guidewire (130) to determine the location of a sensor (not shown) in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

A coupling unit (132) is secured to the proximal end of a navigation guidewire (130). Coupling unit (132) of this example is configured to provide wireless communication of data and other signals between console (116) and navigation guidewire (130). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Navigation guidewire (130) of the present example includes a sensor (not shown) that is responsive to the alternating electromagnetic fields generated by field generators (206). In the present example, the sensor of navigation guidewire (130) comprises at least one wire coil at the distal end of navigation guidewire (130). When such a coil is positioned within an alternating electromagnetic field generated by field generators (206), that the alternating electromagnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end of navigation guidewire (130) within a three-dimensional space (i.e., within the head (H) of the patient (P)). To accomplish this, processor (110) executes an algorithm to calculate location coordinates of the distal end of navigation guidewire (130) from the position related signals of the coil(s) in navigation guidewire (130).

Processor (110) uses software stored in a memory of processor (110) to calibrate and operate system (100). Such operation includes driving field generators (206), processing data from navigation guidewire (130), processing data from operating controls (112), and driving display screen (114). Processor (110) is further operable to provide video in real time via display screen (114), showing the position of the distal end of navigation guidewire (130) in relation to a video camera image of the patient's head (H), a CT scan image of the patient's head (H), and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. By way of example only, display screen (114) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (114).

Those of ordinary skill in the art will recognize that, when navigation guidewire (130) is coupled with a medical instrument, the images provided through display screen (114) may help guide the operator in maneuvering and otherwise manipulating the medical instrument within the patient's head (H) and/or elsewhere within the anatomy of the patient (P).

### II. Exemplary Endoscope with Integral Navigation Sensor

In some instances, it may be desirable to visually confirm where a working element is being placed in addition to knowing the location of an instrument in relation to the CT scan image. In other words, it may be desirable to utilize IGS navigation system (100) in conjunction with other visual aids, such as a video image provided by an endoscope. For instance, during a Eustachian tube dilation, the dilation instrument may need to be carefully navigated to avoid causing trauma to delicate adjacent anatomical structures. When operating on or around delicate anatomical structures, knowing the position of an instrument is in relationship to the CT scan image, while also having endoscopic visualization of the anatomy adjacent to the working device (e.g. a dilation catheter), may help more accurately place the working device in the desired location.

The following examples relate to various endoscope devices having integral navigation sensors configured to for use in IGS navigation system (100) in replacement of, or in addition to guidewire (130). While various examples of endoscopes with integral navigation sensors are described below, it should be understood various combinations or modifications may be made to such endoscopes as would be apparent to one having ordinary skill in the art in view of the teachings herein.

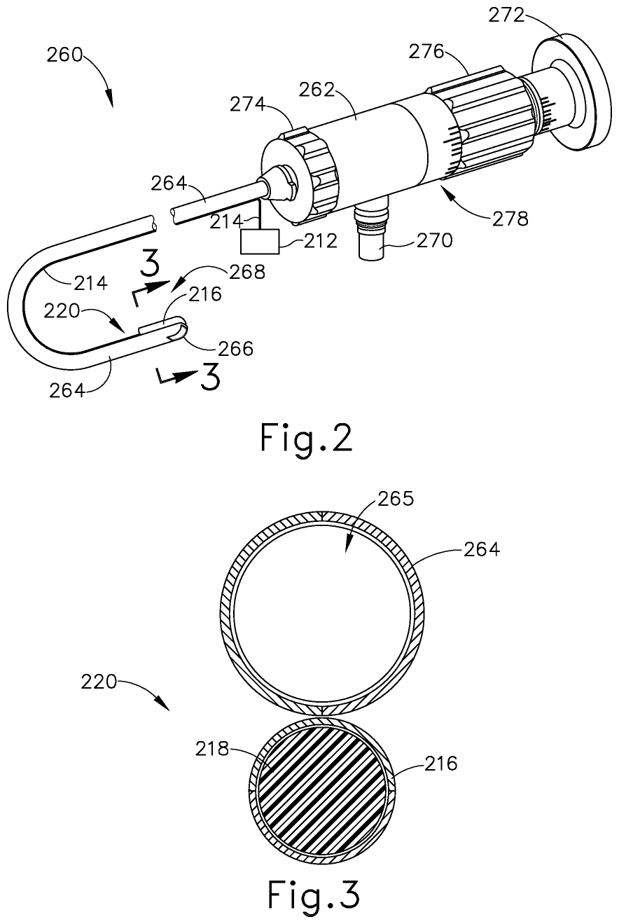

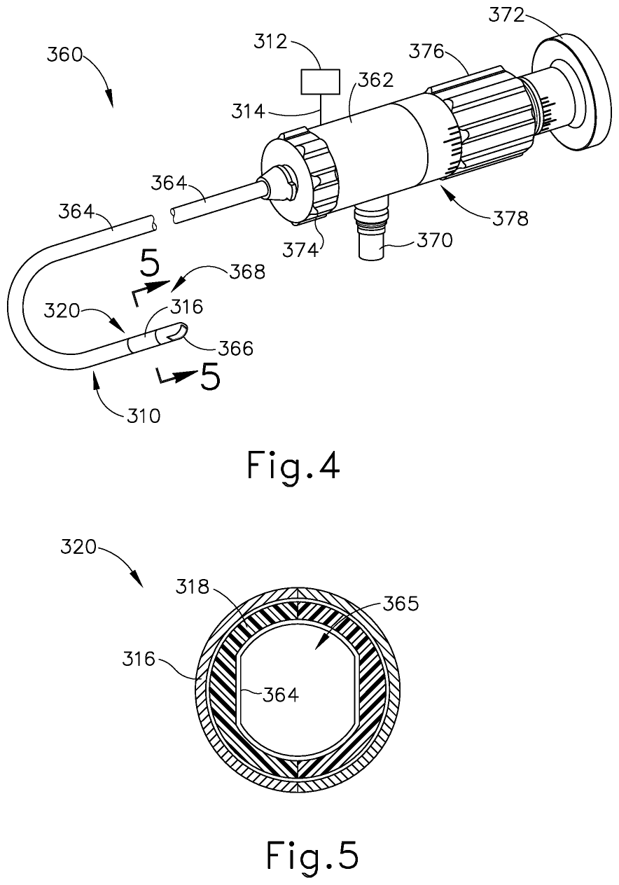

FIG. 2 shows an exemplary endoscope (260) that may be readily incorporated into IGS navigation system (100) described above. Endoscope (260) of the present example comprises a body (262), a flexible shaft (264) extending distally from body (262), and a navigation assembly (210). As will be described in greater navigation assembly (210) is configured to couple with IGS navigation system (100) such that the position of the distal end (268) of flexible shaft (264) is viewable in real time via display screen (114) in relation to a video camera image of the patient's head (H), a CT scan image of the patient's head (H), and/or a computer generated three-dimensional model of the anatomy within and adjacent to the patient's nasal cavity.

Flexible shaft (264) defines a lumen (265) and extends about its own longitudinal axis from body (262), terminating in distal end (268). Flexible shaft (264) is sufficiently flexible such that shaft (264) may bend laterally from a straight longitudinal axis. Therefore, flexible shaft (264) may bend in order to navigate through tortuous paths to reach a desired location within an anatomical structure. Distal end (268) of shaft (264) includes a curved transparent window (266) in the present example. In some other versions, window (266) is not curved. While not shown, a plurality of rod lenses and/or light transmitting fibers may extend within lumen (265) and along the length of shaft (264).

A lens is positioned at the distal end of the rod lenses and a swing prism is positioned between the lens and window (266) in the present example. The swing prism is pivotable about an axis that is transverse to the longitudinal axis of the distal end (268) of shaft (264). The swing prism defines a line of sight that pivots with the swing prism. The line of sight defines a viewing angle relative to the longitudinal axis of the distal end (268) of shaft (264). This line of sight may pivot from approximately 0 degrees to approximately 120 degrees, from approximately 10 degrees to approximately 90 degrees, or within any other suitable range. The swing prism and window (266) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples. Moreover, some variations may omit the swing prism, such that endoscope (260) has only one single, fixed line of sight that is not capable of being adjusted relative to the longitudinal axis of the distal end (268) of shaft (264).

Body (262) of the present example includes a light post (270), an eyepiece (272), a rotation dial (274), and a pivot dial (276). Light post (270) is in communication with the light transmitting fibers within lumen (265) of shaft (264) and is configured to couple with a source of light, to thereby illuminate the site in the patient distal to window (266). Eyepiece (272) is configured to provide visualization of the view captured through window (266) via the optics of endoscope (260). A visualization system (e.g., camera and display screen, etc.) may be coupled with eyepiece (272) to provide visualization of the view captured through window (266) via the optics of endoscope (260). Rotation dial (274) is configured to rotate shaft (264) relative to body (262) about the longitudinal axis of shaft (264). Such rotation may be carried out even while the swing prism is pivoted such that the line of sight is non-parallel with the longitudinal axis of shaft (264). Pivot dial (276) is coupled with the swing prism and is thereby operable to pivot the swing prism about the transverse pivot axis. Indicia (278) on body (262) provide visual feedback indicating the viewing angle. Various suitable components and arrangements that may be used to couple rotation dial (274) with the swing prism will be apparent to those of ordinary skill in the art in view of the teachings herein. By way of example only, endoscope (260) may be configured in accordance with at least some of the teachings of U.S. Pub. No. 2010/0030031, the disclosure of which is incorporated by reference herein. Other suitable forms that endoscope (260) may take will be apparent to those of ordinary skill in the art in view of the teachings herein. By way of example only, some variations of endoscope (260) (e.g., versions that lack a swing prism) may lack rotation dial (274) and/or pivot dial (276) altogether.

Navigation assembly (210) includes a sensor (220) attached to distal end (268) of shaft (264), a coupling unit (212), and a communication wire (214) extending from sensor (220) to coupling unit (212). Coupling unit (212) may be substantially similar to coupling unit (132) described above, with differences elaborated below. Therefore, coupling unit (212) may be coupled with console (116). Coupling unit (212) may couple with console (116) wirelessly, through wired communications such as USB, or via any other suitable means as would be apparent to one having ordinary skill in the art in view of the teachings herein. Various other suitable features and functionality that may be incorporated into coupling unit (212) will be apparent to those of ordinary skill in the art in view of the teachings herein.

Sensor (220) may be substantially similar to sensor (not shown) of navigation guidewire (130) described above, with differences elaborated below. In the current example, sensor (220) includes a coil member (218) encased in a housing (216). Sensor (220) is attached to an external portion of shaft (264) located at distal end (268). In particular, sensor (220) is laterally offset from distal end (268) of shaft (264) such that the sensor is spaced away from the longitudinal axis defined by the adjacent portion of shaft (264). In other words, sensor (220) is attached to shaft (264) such that sensor (220) and adjacent portions of shaft (264) are not coaxial. Communication wire (214) also extends along the external portion of shaft (263) from a proximal portion of sensor (220) all the way to coupling unit (212). In particular, communication wire (214) is in communication with both coil member (218) and coupling unit (212).

While sensor (220) of the present example includes just one single coil member (218) of the present example, some other versions of sensor (220) may include two coil members (218), three coil members (218), or more than three coil members (218). In versions with two or more coil members (218), the different coil members (218) may be oriented along respective axes that are orthogonal to each other or that are otherwise not aligned with each other.

When coil member (218) is positioned within an alternating electromagnetic field generated by field generators (206), the alternating electromagnetic field may generate electrical current in coil member, and this electrical current may be communicated along the communication wire (214) and further to processor (110) via coupling unit (212). Because coil member (218) is fixed relative to shaft (264), this phenomenon may enable IGS navigation system (100) to determine the location of distal end (268) of shaft (264) within a three-dimensional space (i.e., within the head (H) of the patient (P)). To accomplish this, processor (110) executes an algorithm to calculate location coordinates of distal end (268) of shaft (264) from the position related signals of coil member (218). Therefore, during exemplary use, the operator may view the location of distal end (268) of shaft (264) (e.g., in relation to a CT scan or digital model, etc., of the head (H) of the patient (P)) on display screen (114) in real time in accordance with the description herein, and also view a live video feed provided by window (266) of endoscope (260). In other words, the operator may view the live video feed provided by endoscope (260), while also having a reference point of where distal end (268) of shaft (264) is located within head (H) of the patient.

In addition, the light provided via window (266) of endoscope (260) may be used for any other suitable purpose as would be apparent to one having an ordinary skill in the art in view of the teachings herein. For example, the light provided via window (266) may be utilized for a transillumination confirmation of the proper placement of distal end (268) of shaft (264). Alternatively, light provided via window (266) may simply illuminate the field of view for the video images captured by endoscope (260).

FIG. 5 shows another exemplary endoscope (360) that may be readily incorporated into IGS navigation system (100) described above. Endoscope (360) is substantially similar to endoscope (260) described above, with differences elaborated below. Endoscope (360) of the present example comprises a body (362), a flexible shaft (364) extending distally from body (362), and a navigation assembly (310). As will be described in greater navigation assembly (310) is configured to couple with IGS navigation system (100) such that the position of the distal end (368) of flexible shaft (364) is viewable in real time via display screen (114) in relation to a video camera image of the patient's head (H), a CT scan image of the patient's head (H), and/or a computer generated three-dimensional model of the anatomy within and adjacent to the patient's nasal cavity.

Flexible shaft (364) defines a lumen (365) and extends about its own longitudinally axis from body (362) terminating in a distal end (368). Flexible shaft (364) is sufficiently flexible such that shaft (364) may bend laterally from a straight longitudinal axis. Therefore, flexible shaft (364) may bend in order to navigate through tortuous paths to reach a desired location within an anatomical structure. Distal end (368) of shaft (364) includes a curved transparent window (366) in the present example. In some other versions, window (366) is not curved. While not shown, a plurality of rod lenses and light transmitting fibers may extend within lumen (365) and along the length of shaft (364).

A lens is positioned at the distal end of the rod lenses and a swing prism is positioned between the lens and window (366) in the present example. The swing prism is pivotable about an axis that is transverse to the longitudinal axis of the distal end (368) of shaft (364). The swing prism defines a line of sight that pivots with the swing prism. The line of sight defines a viewing angle relative to the longitudinal axis of the distal end (368) of shaft (364). This line of sight may pivot from approximately 0 degrees to approximately 120 degrees, from approximately 10 degrees to approximately 90 degrees, or within any other suitable range. The swing prism and window (366) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples. Moreover, some variations may omit the swing prism, such that endoscope (360) has only one single, fixed line of sight that is not capable of being adjusted relative to the longitudinal axis of the distal end (368) of shaft (364).

Body (362) of the present example includes a light post (370), an eyepiece (372), a rotation dial (374), and a pivot dial (376). Light post (370) is in communication with the light transmitting fibers within lumen (365) of shaft (364) and is configured to couple with a source of light, to thereby illuminate the site in the patient distal to window (366). Eyepiece (372) is configured to provide visualization of the view captured through window (366) via the optics of endoscope (360). A visualization system (e.g., camera and display screen, etc.) may be coupled with eyepiece (372) to provide visualization of the view captured through window (366) via the optics of endoscope (360). Rotation dial (374) is configured to rotate shaft (364) relative to body (362) about the longitudinal axis of shaft (364). Such rotation may be carried out even while the swing prism is pivoted such that the line of sight is non-parallel with the longitudinal axis of shaft (364). Pivot dial (376) is coupled with the swing prism and is thereby operable to pivot the swing prism about the transverse pivot axis. Indicia (378) on body (362) provide visual feedback indicating the viewing angle. Various suitable components and arrangements that may be used to couple rotation dial (374) with the swing prism will be apparent to those of ordinary skill in the art in view of the teachings herein. By way of example only, endoscope (360) may be configured in accordance with at least some of the teachings of U.S. Pub. No. 2010/0030031, the disclosure of which is incorporated by reference herein. Other suitable forms that endoscope (60) may take will be apparent to those of ordinary skill in the art in view of the teachings herein. By way of example only, some variations of endoscope (360) (e.g., versions that lack a swing prism) may lack rotation dial (374) and/or pivot dial (376) altogether.

Navigation assembly (300) includes a sensor (320) attached to distal end (368) of shaft (364), a coupling unit (312), and a communication wire (314) extending from sensor (320) to coupling unit (312). Coupling unit (312) may be substantially similar to coupling unit (132) described above, with differences elaborated below. Therefore, coupling unit (312) may be coupled with console (116). Coupling unit (312) may couple with console (116) wirelessly, through wired communications such as USB, or via any other suitable means as would be apparent to one having ordinary skill in the art in view of the teachings herein. Various other suitable features and functionality that may be incorporated into coupling unit (312) will be apparent to those of ordinary skill in the art in view of the teachings herein.

Sensor (320) may be substantially similar to sensor (not shown) of navigation guidewire (130) described above, with differences elaborated below. In the current example, sensor (320) includes a coil member (318) encased in a housing (316). Sensor (320) is attached to an external portion of shaft (364) located at distal end (368). In particular, coil member (318) wraps around the external portion of shaft (364) such that the distal end of shaft (364) and coil member (318) are coaxial.

While in the current example, coil members (318) wraps around the external portion of shaft (364) and is encased in housing (316), coil member (318) may alternatively wrap around an interior surface of shaft (364) such that shaft (364) acts as a housing for coil member (318). In such examples, coil member (318) may help define corresponding portions of lumen (365). Alternatively, in such examples, an interior coating may surround coil member (318) such that coil member is encased by shaft (364) while the interior coating helps define corresponding portions of lumen (365). While in the current example, coil member (318) wraps around the entire circumference of shaft (364), coil member (318) may only wrap around a sectional portion of the circumference of shaft (364). As yet another merely illustrative example, coil member (318) may be embedded within the sidewall of shaft (364). Of course, any other suitable variations and placements of coil member (318) may be used as would be apparent to one having ordinary skill in the art in view of the teachings herein.

Communication wire (314) from a proximal portion of sensor (320) all the way to coupling unit (312). In particular, communication wire (314) may extend along the interior of shaft (364) within lumen (365), on the exterior of shaft (364), or along shaft (364) as an electrical trace. Communication wire (314) is in communication with both coil member (318) and coupling unit (312).

While sensor (320) of the present example includes just one single coil member (318) of the present example, some other versions of sensor (320) may include two coil members (318), three coil members (318), or more than three coil members (318). In versions with two or more coil members (318), the different coil members (318) may be oriented along respective axes that are orthogonal to each other or that are otherwise not aligned with each other.

When coil member (318) is positioned within an alternating electromagnetic field generated by field generators (306), the alternating electromagnetic field may generate electrical current in coil member, and this electrical current may be communicated along the communication wire (314) and further to processor (110) via coupling unit (312). Because coil member (318) is fixed relative to shaft (364), this phenomenon may enable IGS navigation system (100) to determine the location of distal end (368) of shaft (364) within a three-dimensional space (i.e., within the head (H) of the patient (P)). To accomplish this, processor (110) executes an algorithm to calculate location coordinates of distal end (368) of shaft (364) from the position related signals of coil member (318). Therefore, during exemplary use, the operator may view the location of distal end (368) of shaft (364) (e.g., in relation to a CT scan or digital model, etc., of the head (H) of the patient (P)) on display screen (114) in accordance with the description herein, and also view a live video feed provided by window (366) of endoscope (360). In other words, the operator may view the live video feed provided by endoscope (360), while also having a reference point of where distal end (368) of shaft (364) is located within head (H) of the patient.

In addition, the light provided via window (366) of endoscope (360) may be used for any other suitable purpose as would be apparent to one having an ordinary skill in the art in view of the teachings herein. For example, the light provided via window (366) may be utilized for a transillumination confirmation of the proper placement of distal end (368) of shaft (364). Alternatively, light provided via window (366) may simply illuminate the field of view for the video images captured by endoscope (360).

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) an endoscope, wherein the endoscope comprises: (i) a body, (ii) a shaft extending distally from the body, wherein the shaft comprises a distal portion, and (iii) a window located at the distal portion of the shaft; (b) a navigation sensor positioned at the distal portion of the shaft; and (c) an interface feature, wherein the interface feature is configured to couple the navigation sensor with an image guidance system, wherein the navigation sensor is configured to cooperate with an image guidance system to provide feedback indicating a position of the navigation sensor in three-dimensional space.

### Example 2

The apparatus of Example 1, wherein the shaft includes a flexible member defining a lumen.

### Example 3

The apparatus of any one or more of Examples 1 through 2, wherein the navigation sensor comprises a coil member.

### Example 4

The apparatus of Example 3, wherein the coil member is coupled to an exterior surface of the distal portion of the shaft.

### Example 5

The apparatus of Example 4, wherein the coil member extends around a coil axis that is coaxial with the longitudinal axis of the body.

### Example 6

The apparatus of Example 4, wherein the coil member is not coaxial with the distal portion of the shaft.

### Example 7

The apparatus of any one or more of Examples 1 through 6, further comprising a communication member extending between the navigation sensor and the interface feature.

### Example 8

The apparatus of Example 7, wherein the communication member extends on an exterior surface of the shaft.

### Example 9

The apparatus of any one or more of Examples 1 through 8, wherein the interface feature is configured to couple with the image guidance system wirelessly.

### Example 10

The apparatus of any one or more of Examples 1 through 9, wherein the endoscope further comprises a light post extending from the body.

### Example 11

The apparatus of any one or more of Examples 1 through 10, wherein the endoscope further comprises a pivot dial coupled with the body.

### Example 12

The apparatus of any one or more of Examples 1 through 11, wherein the endoscope further comprises a rotational dial coupled with the body.

### Example 13

The apparatus of any one or more of Examples 1 through 12, wherein the endoscope further comprises an eye piece.

### Example 14

The apparatus of any one or more of Examples 1 through 13, wherein the navigation sensor is covered in a housing.

### Example 15

The apparatus of any one or more of Examples 1 through 14, wherein the navigation sensor consists of a single axis sensor.

### Example 16

An apparatus comprising: (a) an endoscope comprising: (i) a body, (ii) a shaft extending distally from the body, wherein the shaft comprises a distal portion, (iii) a window fixed at the distal portion of the shaft, wherein the window is configured to transmit light and transmit an image, and (iv) a visualization feature configured to provide visualization of the image transmitted through the window; and (b) a navigation assembly, wherein the navigation assembly comprises: (i) a navigation sensor fixed to the distal portion of the shaft, wherein the navigation sensor is configured to cooperate with an image guidance system to generate an electrical signal in response to an external magnetic field, thereby indicating a position of the navigation sensor within the magnetic field, and (ii) an interface feature configured to couple the navigation sensor with an image guidance system.

### Example 17

The apparatus of Example 16, wherein the navigation assembly is proximal relative to the window.

### Example 18

The apparatus of any one or more of Examples 1 through 17, wherein the navigation assembly further comprises a communication wire extending between the navigation sensor and the interface feature.

### Example 19

An apparatus comprising: (a) an endoscope, wherein the endoscope comprises: (i) a body, and (ii) a shaft extending distally from the body, wherein the shaft comprises a distal portion; and (b) a navigation assembly, wherein the navigation assembly comprises: (i) a coil fixed to the distal portion of the shaft, wherein the coil is configured to cooperate with an image guidance system to generate an electrical signal in response to an external magnetic field, thereby indicating a position of the navigation sensor within the magnetic field, (ii) an interface feature configured to couple the navigation sensor with an image guidance system, and (iii) an electrical conduit coupled with the coil and the interface feature, wherein the electrical conduit is configured to transfer the electrical signal from the coil to the interface feature.

### Example 20

The apparatus of Example 19, wherein the coil wraps around the distal portion of the shaft along a coil axis, wherein the distal portion of the shaft defines a second axis, wherein the coil axis and the second axis are coaxial.

### IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) an endoscope, wherein the endoscope comprises:
(i) a body,
(ii) a shaft extending distally from the body, wherein the shaft comprises a distal portion, and
(iii) a window located at the distal portion of the shaft;
(b) a navigation sensor positioned at the distal portion of the shaft; and
(c) an interface feature, wherein the interface feature is configured to couple the navigation sensor with an image guidance system, wherein the navigation sensor is configured to cooperate with an image guidance system to provide feedback indicating a position of the navigation sensor in three-dimensional space.

2. The apparatus of claim 1, wherein the shaft includes a flexible member defining a lumen.

3. The apparatus of claim 1, wherein the navigation sensor comprises a coil member.

4. The apparatus of claim 3, wherein the coil member is coupled to an exterior surface of the distal portion of the shaft.

5. The apparatus of claim 4, wherein the coil member extends around a coil axis that is coaxial with the longitudinal axis of the body.

6. The apparatus of claim 4, wherein the coil member is not coaxial with the distal portion of the shaft.

7. The apparatus of claim 1, further comprising a communication member extending between the navigation sensor and the interface feature.

8. The apparatus of claim 7, wherein the communication member extends on an exterior surface of the shaft.

9. The apparatus of claim 1, wherein the interface feature is configured to couple with the image guidance system wirelessly.

10. The apparatus of claim 1, wherein the endoscope further comprises a light post extending from the body.

11. The apparatus of claim 1, wherein the endoscope further comprises a pivot dial coupled with the body.

12. The apparatus of claim 1, wherein the endoscope further comprises a rotational dial coupled with the body.

13. The apparatus of claim 1, wherein the endoscope further comprises an eye piece.

14. The apparatus of claim 1, wherein the navigation sensor is covered in a housing.

15. The apparatus of claim 1, wherein the navigation sensor consists of a single axis sensor.

16. An apparatus comprising:
(a) an endoscope comprising:
(i) a body,
(ii) a shaft extending distally from the body, wherein the shaft comprises a distal portion,
(iii) a window fixed at the distal portion of the shaft, wherein the window is configured to transmit light and transmit an image, and
(iv) a visualization feature configured to provide visualization of the image transmitted through the window; and
(b) a navigation assembly, wherein the navigation assembly comprises:
(i) a navigation sensor fixed to the distal portion of the shaft, wherein the navigation sensor is configured to cooperate with an image guidance system to generate an electrical signal in response to an external magnetic field, thereby indicating a position of the navigation sensor within the magnetic field, and
(ii) an interface feature configured to couple the navigation sensor with an image guidance system.

17. The apparatus of claim 16, wherein the navigation assembly is proximal relative to the window.

18. The apparatus of claim 16, wherein the navigation assembly further comprises a communication wire extending between the navigation sensor and the interface feature.

19. An apparatus comprising:
(a) an endoscope, wherein the endoscope comprises:
(i) a body, and
(ii) a shaft extending distally from the body, wherein the shaft comprises a distal portion; and
(b) a navigation assembly, wherein the navigation assembly comprises:
(i) a coil fixed to the distal portion of the shaft, wherein the coil is configured to cooperate with an image guidance system to generate an electrical signal in response to an external magnetic field, thereby indicating a position of the navigation sensor within the magnetic field,
(ii) an interface feature configured to couple the navigation sensor with an image guidance system, and
(iii) an electrical conduit coupled with the coil and the interface feature, wherein the electrical conduit is configured to transfer the electrical signal from the coil to the interface feature.

20. The apparatus of claim 19, wherein the coil wraps around the distal portion of the shaft along a coil axis, wherein the distal portion of the shaft defines a second axis, wherein the coil axis and the second axis are coaxial.
